(19) United States
(12) Patent Application Publication
(10) Pub. No.: US 2026/0355742 A1
(43) Pub. Date: Nov. 12, 2026

(54) MICROFLUIDIC DEVICE FOR CELL COMPRESSION

(71) Applicant: The University of York, York (GB)

(72) Inventors: Rasha Saber Mohamed Mahmoud REZK, York (GB); David Geoffrey KENT, York (GB)

(21) Appl. No.: 19/129,193

(22) PCT Filed: Nov. 10, 2023
(86) PCT No.: PCT/EP2023/081505

§ 371 Date: May 12, 2025

(30) Foreign Application Priority Data
Nov. 11, 2022 (GB) 2216849.6

(51) Int. Cl. B01L 3/00

(52) U.S. Cl. CPC B01L 3/502761; B01L 3/502738; B01L 2200/0621; B01L 2200/0668; B01L 2400/0481; B01L 2400/0487

(57) ABSTRACT

The disclosure concerns a microfluidic device suitable for mechanically compressing cells without the necessity of adhering cells onto a substrate, methods for mechanically compressing cells and mechanically compressed cells obtained by the method are also disclosed.

### FIELD OF THE INVENTION

The disclosure concerns a microfluidic device suitable for mechanically compressing cells without the necessity of adhering cells onto a substrate, methods for mechanically compressing cells, assessing mechanically compressed cells and mechanically compressed cells obtained by the method are also disclosed.

### BACKGROUND OF THE INVENTION

Mechanical stimulation influences the physiological response of cells resulting in biochemical signalling. All cells experience a variety of different mechanical forces such as shear, stretch or compression forces. High compression forces can for example be found within a tumour environment or on epithelial cells because of airway restriction in asthma patients.

Modelling of pathological disease states is vital for understanding physiological responses within the cell and aids improved drug development, however, mimicking the natural cell environment is also important in other areas such for example improving laboratory methods such as cell transformation or cell propagation.

For example, the standard treatment for blood cancers (such as leukaemia and lymphoma), and other life-threatening blood diseases (e.g. sickle cell anaemia), require the transplantation of healthy stem cells derived from bone marrow harvest or apheresis from a genetically matched donor, usually a sibling. Only 30% of patients can find a fully matched donor within their family. Consequently, we need to identify an alternative source of stem cells for most patients. Therefore, a long sought-after goal is to identify and recapitulate conditions capable of maintaining and expanding functional blood stem cells (such as haematopoietic stem cells (HSCs)) outside the body. Despite our improved understanding of the chemical and molecular modifiers of HSC expansion, researchers have yet to produce a large supply of “on-demand” blood cell types.

In adults, blood cells are derived from HSCs which reside in the bone marrow. During development, blood cells are subjected to various forces such as the frictional force generated by blood flow, or compressive stresses generated from their neighbouring cells. However, there is currently no tool that can compress a population of live cells in suspension for prolonged periods and subsequently retrieve them for functional and molecular analysis.

At present, non-adherent single cells can only be deformed by extensional flow [2] through microfluidic devices, to measure their mechanical properties in a cytometer [3]. Such chips subject individual cells to shear and compressive stresses simultaneously for a very short amount of time (a few seconds) and rely on the mechanical properties of the individual cell (deformability). Tools to explore the biomechanics of non-adherent cells such as HSCs which would help to improve our understanding of ex-vivo cell expansion are currently non-existing. Microfluidic devices are manifold and have a variety of applications with biomedical and biological diagnostic and research. They have the potential to reduce cost of reagent and time of experiment, allow extraction and detection of protein, nucleic acids, enzymes and metabolites and have wide applications in live imaging and cellular migration.

The disclosure concerns a microfluidic device which allows the controlled application of mechanical forces to a population of non-adherent cells for extended periods of time with subsequent retrieval of said compressed cells.

### STATEMENT OF THE INVENTION

According to an aspect of the invention there is provided a microfluidic device comprising a compression chamber (4,8) comprising a chamber (8) and a compression membrane (4) adapted to contain and compress cells contained therein, wherein said compression chamber (4, 8) is provided with at least first and second channels (9, 10) in fluid connection with said compression chamber (4, 8) wherein said first channel (9) is provided with an inlet (2a) at or near the end of said first channel (9) for receiving cells and further comprising a first valve (3) to regulate the flow of cells from said inlet (2a) to said compression chamber (4, 8) via said first channel (9) and wherein said second channel (10) is provided with an outlet (6a) at or near the end of said second channel (10) to regulate, when in use, the exit of compressed cells contained in said compression chamber (4, 8) and further comprising a second valve (5) to regulate the flow of compressed cells from said compression chamber (4,8) via said second channel (10) wherein said first and second valves (3, 5) are independently regulatable to control the ingress and egress of cells respectively into and out of said compression chamber (4, 8).

According to an aspect of the invention there is provided a microfluidic device comprising a compression chamber (4,8) comprising a chamber (8) and a compression membrane (4) adapted to contain and compress cells contained therein, wherein said compression chamber (4, 8) is provided with at least first and second channels (9, 10) in fluid connection with said compression chamber (4, 8) wherein said first channel (9) is provided with an inlet (2a) at or near the end of said first channel (9) for receiving cells and further comprising a first valve (3) to regulate the flow of cells from said inlet (2a) to said compression chamber (4, 8) via said first channel (9) and wherein said second channel (10) is provided with an outlet (6a) at or near the end of said second channel (10) to regulate, when in use, the exit of compressed cells contained in said compression chamber (4, 8) and further comprising a second valve (5) to regulate the flow of compressed cells from said compression chamber (4,8) via said second channel (10) wherein said first and second valves (3, 5) are independently regulatable to control the ingress and egress of cells respectively into and out of said compression chamber (4, 8) and wherein there is provided a fifth channel (13) in fluid connection with the first (9) and second channel (10) to receive a fluid and/or a fluid comprising cells during compression.

The microfluidic device of the invention enables the application of pressure on, for example, non-adherent cells. The skilled artisan will appreciate that the dimensions of the microfluidic device are based on the size of the cells to be compressed.

Typically, the width and height of the channels and compression chamber is between 20% 80% larger than the cell size.

Preferably, the width and height of the channels and compression chamber is between 20% 40% larger than the cell size.

In a preferred embodiment of the invention of the invention said microfluidic device comprises a silicone-based polymer.

In a preferred embodiment of the invention said microfluidic device comprises poly(dimethylsiloxane) (PDMS).

In a preferred embodiment of the invention said device comprises PDMS at a ratio of 5:1 of PDMS to catalyst.

In a preferred embodiment of the invention said device comprises PDMS at a ratio of 20:1 of PDMS to catalyst.

In a further preferred embodiment of the invention said device comprises two layers of PDMS wherein the first layer comprises a ratio of 5:1 of PDMS to catalyst and the second layer comprises a ratio of 20:1 of PDMS to catalyst.

In a preferred embodiment of the invention said membrane has an upper and lower surface and a space there between the upper and lower membrane for receiving a fluid.

In a preferred embodiment of the invention said membrane has an upper and lower surface and a space exists between the upper and lower membrane for receiving a fluid.

In the context of this invention the membrane forms a pneumatic microvalve which expands when receiving a fluid such as gas or air and exerts pressure onto the chamber and the cells therein. Alternatively, the microvalve can be filled with a liquid and expands by receiving a fluid such as air or gas to push the liquid further into the membrane to deflect the membrane and exert pressure onto the chamber. An example of a pneumatic microvalve is a Quake valve. In the context of the invention the term “valves” describes a device which regulates or controls the flow of a fluid in a channel by opening, closing, partially opening and partially closing the valve. Preferably, said valves are formed by an upper and lower membrane wherein a space exists between the upper and lower layer and is controlled by receiving a fluid such as gas or air, expanding and subsequently exerting pressure onto the channels to open, closing or partially closing the channels to control or regulate the flow of the fluid in the channels. For example, a Quake valve can be used.

In a preferred embodiment of the invention said membrane comprises silicone, for example, poly(dimethylsiloxane) (PDMS).

In the context of this invention the microfluidic device comprises two layers wherein the second or lower layer comprises the chamber (8), the first and second and fifth channels (9, 10, 15), the inlet (2a) and the outlet (6a) and the first or upper layer comprises the valves (3, 5) and membrane (4).

The valves and membrane are as described preferable pneumatic valves such as Quake valves which can be independently controlled to open and close the channels to at least partially stop the flow of fluids in the channels and exert pressure to the chamber.

The microfluidic device is conveniently placed onto a solid support such as a glass slide. The second or lower layer is termed flow layer and the first or upper layer comprising the membrane and valves is the control layer.

In a preferred embodiment of the invention said membrane (4) has a width and the width of the membrane exceeds the width of the chamber (8).

In a preferred embodiment of the invention said chamber (8) has a length and the length of the chamber exceeds the length of the membrane (4).

In a preferred embodiment of the invention the length of the compression chamber (4,8) is defined by the distance between the first and second valves (3 and 5) and wherein the length of the membrane (4) is less than the distance between the first and second valves (3,5) to allow optimal closure of the first and second valves.

For example, the membrane covers at least 90, 91, 92, 93, 94, 95, 96, 97, 98 and 99% of the length of the chamber.

The width of the membrane (4) exceeds the width of the chamber (8) to exert uniform compression on all areas within the chamber (8).

In a preferred embodiment of the invention said chamber has a length of 2120 μm+/−10%. In a preferred embodiment of the invention said chamber has a length of 2060 μm+/−10%. In a preferred embodiment of the invention said chamber has a width of between 40 μm+/−10% to 400 μm+/−10%.

In a further preferred embodiment of the invention said chamber has a width of between 50-300 μm, more preferably between 100-250 μm, and even more preferably between 150-200 μm.

In a further preferred embodiment of the invention said chamber has a width of 40 μm+/−10%. In a preferred embodiment of the invention said chamber has a width of 100 μm+/−10%.

In a preferred embodiment of the invention said membrane is between 100-500 μm in width, more preferably between 200-500 μm in width.

In a preferred embodiment of the invention said membrane has a length of 2120 μm+/−10%. In a preferred embodiment of the invention said membrane is 2060 μm in length.

In a preferred embodiment of the invention said membrane is at least 90% of 2060 μm in length.

In a further preferred embodiment of the invention said membrane is at least 91, 92, 93, 94, 95, 96, 97, 98 or 99% of 2060 μm in length.

In a further preferred embodiment of the invention said first channel comprises a further valve (2) to regulate the flow from the first inlet (2a) into the first channel (9).

In a further preferred embodiment of the invention said first channel comprises a further valve (6) to regulate the flow from the second channel (10) into the first outlet (6a).

In a further preferred embodiment of the invention said second channel comprises a further valve (6) to regulate the flow towards the second channel (10) into the first outlet (6a).

In an alternative preferred embodiment of the invention said first channel (9) is in fluid connection with a third channel (11) and provided with a second inlet (1a) at or near the end of said third channel (11) for receiving cells or liquids.

In an alternative preferred embodiment of the invention said first channel (9) is in fluid communication with a third channel (11) which is provided with a second inlet (1a) at or near the end of said third channel (11) for receiving cells or liquids.

In a preferred embodiment of the invention said third channel (11) is provided with a valve (1) to regulate the flow from the second inlet (1a) to the first channel (9) and compression chamber (8,4).

In a preferred embodiment of the invention said second channel (10) is in fluid connection with a fourth channel (12) and provided with a second outlet (7a) at or near the end of said fourth channel (12).

In a preferred embodiment of the invention said second channel (10) is in fluid communication with a fourth channel (12) which is provided with a second outlet (7a) at or near the end of said fourth channel (12).

In a preferred embodiment of the invention said fourth channel (12) is provided with a valve (7) to regulate the flow from the compression chamber (4,8) and the second channel (12) to the second outlet (7a).

In a preferred embodiment of the invention there is provided a fifth channel (13) in fluid connection with the first (9) and second channel (10) to receive a fluid and/or a fluid comprising cells during compression.

Preferably said fifth channel (13) is positioned substantially in parallel with the compression chamber (4,8).

The fifth channel (13) allows receiving and redirection of the liquid medium comprising the cells during compression of the cells due to the non-compressible nature of a liquid.

Preferably, the second or lower layer comprising the chamber (8), the first, second and fifth channels (9, 10, 13), the inlet (2a) and the outlet (6a) and comprises further the third (11) and fourth (12) channel, the second inlet (1a) and second (7a) outlet and the first or upper layer comprising the valves (3, 5) and membrane (4) comprises further valves (2, 6, 1 and 7).

In a further preferred embodiment of the invention said first, second, third, fourth and fifth channels are 2 μm to 35 μm in height, or preferably between 2 μm-25 μm in height.

In a preferred embodiment of the invention said membrane is at least 90% of 2060 μm in length.

In a further preferred embodiment of the invention said first, second, third, fourth and fifth channels are 16 μm±5 μm in height.

Preferably, said first, second, third, fourth and fifth channels are 2, 3, 4, 5, 6, 7, 8, 9, 10, 11, 12, 13, 14, 15, 16, 17, 18, 19, 20, 21, 22, 23, 24 or 25 μm in height.

In a preferred embodiment of the invention said first, second, third, fourth and fifth channels are 100 μm+/10 % in width.

In a preferred embodiment of the invention said first, second, third, fourth and fifth channels are 100 μm+/10 % in width and 16 μm±5 μm in height.

In a preferred embodiment of the invention said first, second, third, fourth and fifth channels are 100 μm+/10 % in width and 2 μm ±25 μm in height.

In a preferred embodiment of the invention said first, second, third, fourth and fifth channels are 100 μm+/10 % in width and 2, 3, 4, 5, 6, 7, 8, 9, 10, 11, 12, 13, 14, 15, 16, 17, 18, 19, 20, 21, 22, 23, 24 or 25 μm in height.

In a preferred embodiment of the invention said chamber is 16 μm±5μm in height.

In a preferred embodiment of the invention said chamber is 25 μm±2μm in height.

In a preferred embodiment of the invention said chamber is 40 μm±5μm in height.

In a preferred embodiment of the invention said chamber is 4 μm±1μm in height.

In a preferred embodiment of the invention said chamber is 100 μm±/10 % in width and 16 μm±5 μm in height.

In an alternative preferred embodiment of the invention said chamber is 200-300 μm in width and 30 μm in height.

In a further alternative embodiment of the invention said chamber is 40 μm±/10% in width and 2-4 μm in height.

In a preferred embodiment of the invention the height of the first, second, third, fourth, fifth channel and the chamber is the same.

In a preferred embodiment of the invention said chamber is 2, 3, 4, 5, 6, 7, 8, 9, 10, 11, 12, 13, 14, 15, 16, 17, 18, 19, 20, 21, 22, 23, 24 or 25 μm in height.

In a preferred embodiment of the invention said compression chamber is not adapted by the provision of a cell attachment surface, for example said compression chamber is not coated with cell attachment proteins such as fibronectin.

In a preferred embodiment of the invention said device comprises cells.

In a further preferred embodiment said cells are a heterogenous population of cells.

In a further preferred embodiment said cells are a homogenous population of cells.

Preferably, said microfluidic device is linked to a visual display to observe the cells during compression.

According to an aspect of the invention there is provided a method for the compression of cells comprising the steps:
i) providing a microfluidic device comprising a compression chamber comprising a chamber (8) and a compression membrane (4) adapted to contain and compress cells contained therein, wherein said compression chamber (4, 8) is provided with at least first and second channels (9, 10) in fluid connection with said compression chamber (4. 8) wherein said first channel (9) is provided with an inlet (2a) at or near the end of said first channel (9) for receiving cells and further comprising a first valve (3) to regulate the flow of cells from said inlet (2a) to said compression chamber (4, 8) via said first channel (9) and wherein said second channel (10) is provided with an outlet (6a) at or near the end of said second channel (10) to regulate, when in use, the exit of compressed cells contained in said compression chamber (4, 8) and further comprising a second valve (5) to regulate the flow of compressed cells from said compression chamber (4,8) via said second channel (10) wherein said first and second valves (3, 5) are independently regulatable to control the ingress and egress of cells respectively into and out of said compression chamber (4, 8) and a fifth channel (13) in fluid connection with the first (9) and second channel (10) to receive a fluid and/or a fluid comprising cells during compression;
ii) feeding a cell sample to be compressed in said first channel (9) via said inlet (2a) into said compression chamber (4, 8); and partially close said second valve (5) to maintain flow of fluids but prevent movement of cells;
iii) partially closing valve (2);
iv) partially close said first valve (3) to maintain flow of fluids but prevent movement of cells thereby containing said cell sample in said compression chamber (4, 8);
v) actuating said compression membrane (4) of said chamber (8) thereby deflecting said compression membrane (4) and imparting compression force on said chamber (8) containing said cell sample to compress cells contained therein;
vi) opening valve (2) and feeding buffer into said first channel (9) via said inlet (2a) into said compression chamber (4, 8);
vii) open said second valve (5), actuating said membrane (4) of said compression chamber (4, 8) thereby deflecting said membrane and removing compression forces on said chamber (8) applied in step v) and open said first valve (3) to allow egress of compressed cells from said compression chamber into said second channel (10); and
viii) collecting said compressed cells via outlet (6a).

Preferably said fifth channel (13) is positioned substantially in parallel with the compression chamber (4,8).

In a preferred embodiment said first channel in i) comprises a further valve (2) to regulate the flow from the first inlet (2a) into the first channel (9).

The valves are partially closed during compression so that the cells are retained in the chamber and fluid is allowed to flow during compression.

In a preferred method of the invention said microfluidic device comprises further a third channel (11), wherein the third channel (11) in fluid connection with said first channel (9) and is provided with valve (1) and a second inlet (1a) at or near the end of said third channel (11) and wherein said valve (1) regulates the flow from the second inlet (1a) to the first microfluidic channel (9) and compression chamber (4,8) and
a fourth channel (12), wherein the fourth channel (12) in fluid connection with said second channel (10) and is provided with valve (7) and a second outlet (7a) at or near the end of said fourth channel (12) and wherein valve (7) regulates the flow from the compression chamber (4,8) and second microfluidic channel (10) via the fourth channel (12) to the second outlet (7a),
and wherein step ii) includes closing of valve (1) and (7) prior feeding a cell sample to be compressed
and wherein step iv) after partially closing valve (3) further comprises opening valve (1) and feeding buffer via inlet (1a) in said third (11), first (9) and fifth (13) channel to allow removal of non-compressed cells via the second cannel (10) and first outlet (6a) and wherein valve (7) is closed; and wherein step viii) alternatively comprises closing valve (6) and opening valve (7) to collect compressed cells via the second outlet (7a).

In a preferred method of the invention said microfluidic device further comprises further a third channel (11), wherein the third channel (11) is in fluid communication with said first channel (9) and is provided with a third valve (1) and a second inlet (1a) at or near the end of said third channel (11) and wherein said third valve (1) regulates the flow from the second inlet (1a) to the first microfluidic channel (9) and compression chamber (4,8) and
a fourth channel (12), wherein the fourth channel (12) in fluid communication with said second channel (10) and is provided with a fourth valve (7) and a second outlet (7a) at or near the end of said fourth channel (12) and wherein said fourth valve (7) regulates the flow from the compression chamber (4,8) and second microfluidic channel (10) via the fourth channel (12) to the second outlet (7a),
and wherein step ii) includes closing of valve (1) and (7) prior feeding a cell sample to be compressed
and wherein step iv) after partially closing valve (3) further comprises opening valve (1) and feeding buffer via inlet (1a) in said third (11), first (9) and fifth (13) channel to allow removal of non-compressed cells via the second cannel (10) and first outlet (6a) and wherein valve (7) is closed; and wherein step viii) alternatively comprises closing valve (6) and opening valve (7) to collect compressed cells via the second outlet (7a).

Preferably said step v) comprises further the step v1: actuating said compression membrane (4) of said compression chamber (4,8) thereby deflecting said membrane and removing compression forces on said chamber (8) followed by actuating said compression membrane (4) of said chamber (8) thereby deflecting said membrane and imparting compression forces on said chamber (8) to provide cyclic compression on said cells.

The term “deflecting” in the context of the invention means bending the membrane towards the channel or chamber to impart compression forces onto the cells or bending the membrane away from the channel to remove compression forces from the cells.

Preferably step v1 includes opening of valve 3 after deflecting the membrane to remove compression forces on said chamber and closing the valve 3 prior to actuating said membrane of said chamber to deflect the membrane to impart compression forces on said chamber.

Preferably during step v1 valves 1, 2, 5 and 7 are partially closed.

Preferably said step v1 is repeated 1, 2, 3, 4, 5, 6, 7, 8, 9 or 10 times and even more preferably 5 times Preferably said deflection of the membrane (4) to impart compression onto the chamber in step v1 is for a length of 1, 2, 3, 4, 5, 6, 7, 8, 9, 10, 11, 12, 13, 14, 15, 16, 17, 18, 19, 20, 25, 30 min.

Preferably said deflection of the membrane (4) to remove compression onto said chamber in step v1 is for 1, 2, 3, 4, 5, 6, 7, 8, 9, 10, 11, 12, 13, 14, 15, 16, 17, 18, 19, 20, 25, 30 min.

Preferably, the time between actuating said compression membrane (4) of said chamber (8) to deflect said membrane and removing compression forces on said chamber (8) and subsequently deflecting said membrane to impart compression forces onto said chamber to provide cyclic compression on said cells is 1, 2, 3, 4 or 5 min.

Preferably, said applied compression force is increased during each compression cycle.

In a preferred method of the invention said compression force is sequentially increased from a starting force of 0.1 kPa up to between 100-250 kPa and preferably from 0.1 kPa up to between 150-190 kPa and even more preferable between 0.1 to 100 kPa.

In an alternative preferred method of the invention said compression force is sequentially increased from a starting force of 0.1 kPa up to 50 kPa and preferably from 0.1 kPa up to 35 kPa and even more preferable between 0.1 to 25 kPa.

Preferably said cell sample comprises cells in a cell suspension.

It is understood by the skilled person that the cells in a cell sample are suspended in biological buffers and cell culture media.

In a preferred embodiment said cells are a suspension of heterogeneous cells.

In an alternative preferred embodiment said cells are a suspension of homogeneous cells.

In a preferred method of the invention said cells are non-adherent cells.

In a preferred method of the invention said cells are eukaryotic cells.

In a preferred method of the invention said cells are prokaryotic cells.

In a preferred method of the invention said cells are prokaryotic cells are gram positive or gram negative cells.

Preferably said prokaryotic cells are bacterial cells selected from the group consisting of Escherichia coli or Staphylococcus aureus.

In a preferred method of the invention said cells are mammalian cells, preferably human or mouse cells.

In a preferred method of the invention said cells are mature cells.

Preferably, said mature cells are selected from the group: a nerve cell; a muscle cell (cardiomyocyte); a liver cell, e.g. hepatocyte; a kidney cell; a blood cell (e.g. CD4+ lymphocyte, CD8+ lymphocyte; a pancreatic β cell; an epithelial cell (e.g., lung, gastric); an endothelial cell and a CAR T-cell.

In a preferred method of the invention said cell is an immature stem/progenitor cell.

Preferably said cells are progenitor cells selected from the group consisting of: Short-term HSCs (ST-HSCs); multi-potent progenitors (MPPs); lympho-myeloid primed progenitors (LMPPs); common myeloid progenitors (CMPs); granulocyte-macrophage progenitors (GMPs); or megakaryocyte-erythrocyte progenitors (MEPs).

In a preferred method of the invention said immature cell is a haematopoietic stem cell (HSC). In a preferred method of the invention said cells are provided to the first channel under ii) at between 0.1 μl/min to 2.1 μl/min, or preferably between 1 to 1.5 μl/min or between 1 to 1.8 μl/min.

The invention said cells are provided to the first channel under ii) at 0.6, 1, 1.5, 1.8 and 2.1 μl/min.

In a preferred method of the invention said cells are provided to the first channel under ii) for 1 to 5 min, or preferably for 1, 2, 3, 4 or 5 min.

In a preferred method of the invention said compression force is between 5-30 kPa, preferably between 10-28.5 kPa more preferably between 12-20 kPa and even more preferable between 20-28 kPa.

In a preferred method of the invention said compression force is selected from the group consisting of 20, 21, 22, 22.5, 23, 23.5, 24, 24.5, 25, 25.5, 26, 26.5, 27, 27.5, 28, and 28.5, 29 and 30 kPa.

In a preferred method of the invention said compression force is selected from the group consisting of 22.5 kPa, 25 kPa, 28 kPa.

Preferable said compression force is applied for between 1-10 min.

Preferable said compression force is applied for between 10-60 min and more preferably between 20-50 or 30-40 min.

Preferable said compression force is applied for 1, 2, 3, 4, or 5 min.

Preferable said compression force is applied for 5, 10, 15, 20, 25, 30, 35, 40, 45, 50, 55 or 60 min.

According to an aspect of the invention there are cells obtained by the method according to the invention.

In a preferred embodiment of the invention said cells are mammalian cells, preferably human and mouse cells.

In a preferred embodiment of the invention, said cells are mature cells.

Preferably, said mature cells are selected from the group: a nerve cell; a muscle cell (cardiomyocyte); a liver cell, e.g. hepatocyte; a kidney cell; a blood cell (e.g. CD4+ lymphocyte, CD8+ lymphocyte; a pancreatic β cell; an epithelial cell (e.g., lung, gastric); a an endothelial cell and a CAR T-Cell.

In a preferred method of the invention said cell is an immature stem/progenitor cell.

In a preferred method of the invention said progenitor cells are selected from the group consisting of: Short-term HSCs (ST-HSCs); multi-potent progenitors (MPPs); lympho-myeloid primed progenitors (LMPPs); common myeloid progenitors (CMPs); granulocyte-macrophage progenitors (GMPs); or megakaryocyte-erythrocyte progenitors (MEPs).

In a preferred embodiment of the invention, the immature cell is a haematopoietic stem cell. In a preferred method of the invention said microfluidic device is linked to a visual display to monitor the cells during compression.

In a preferred method of the invention said microfluidic device is linked to a graphical user interface to control the compression and visually monitor the cells during compression.

According to an aspect there are provided compressed cells obtained by the method of the invention.

According to an aspect of the invention there is provided the use of the microfluidic device for the delivery of biological materials into a cell.

In a preferred embodiment said biological material is selected from the group consisting of: Peptides, proteins, protein complexes, lipid nanoparticles, extracellular vesicles, exosome and nucleic acid molecules such as DNA such as vector DNA, genomic DNA, antisense oligonucleotides, RNA such as mRNA, siRNA.

Preferably, said biological material is selected from the group consisting of peptides, proteins, protein complexes in combination with nucleic acid molecules include DNA such as vector DNA, genomic DNA, antisense oligonucleotides, RNA such as mRNA, siRNA.

According to an aspect of the invention there is provided the use of the microfluidic device for cell lysis.

According to an aspect of the invention there is provided the use of the microfluidic device for altering cell maturation.

According to an aspect of the invention there is provided the use of the microfluidic device for measuring cell membrane integrity.

Cell membrane integrity can be measured by monitoring the deformation of the cell membrane under pressure up to cell lysis.

According to an aspect of the invention there is provide a method for inducing maturation of an immature cell into a mature cell comprising the method according to the invention.

Preferably said immature cell is a haematopoietic stem or progenitor cell.

Preferably said mature cell is selected from the group consisting of neutrophils, macrophages, monocytes, basophils, eosinophils, B and T lymphocytes, innate lymphoid cells, dendritic cells, megakaryocytes, or platelets.

Preferably said immature cells are compressed at between 20-28 kPa for between 30 to 60 min.

Preferably said immature cells are compressed for 30 mins at 22.5 kPa, 25 kPa or 28 kPa and for 60 mins at 22.5 kPa or 20 kPa.

According to an aspect of the invention there is provided a method for delivering biological materials into cells comprising the method according to the invention, wherein the method comprises alternative step ii) feeding a cell sample to be compressed and biological material to be delivered into the cell comprised in the cell sample in said first channel (9) via said inlet (2a) into said compression chamber (4, 8); and partially close said second valve (5) to maintain flow of fluids but prevent movement of cells.

According to an aspect of the invention there is provide a method for delivering biological material into cells comprising the method according to the invention, wherein said cell sample comprises biological material to be delivering into the cell.

### Preferably Said Cell Is a Haematopoietic Stem Cell.

Preferably said mature cell is selected from the group consisting of neutrophils, macrophages, monocytes, basophils, and eosinophils, B and T lymphocytes, innate lymphoid cells, dendritic cells, megakaryocytes, or platelets.

Preferably said cell is human cell line HL60.

Preferably said cells are compressed at between 10-25 kPa, preferably between 15-17.5 kPa. Preferably said cells are compressed for between 3 to 10 mins.

Preferably said cells are compressed at 15 kPa for 19 min, or alternatively at 17.5 kPa for 10 min.

Preferably said membrane is deflected to remove compression forces in step v1 for between 3 to 10 mins.

Preferably said step v1 of the method is repeated, 1, 2, 3, 4, 5, 6, 7, 8, 9 or 10 times.

Preferably said compression force is sequentially increased from a starting force of 0.1 kPa up to between 100-250 kPa and preferably from 0.1 kPa up to between 150-190 kPa and even more preferable between 0.1 to 100 kPa, or alternatively said compression force is sequentially increased from a starting force of 0.1 kPa up to 50 kPa and preferably from 0.1 kPa up to 35 kPa and even more preferable between 0.1 to 25 kPa.

Preferably, said biological material is selected from the group consisting of peptides, proteins, protein complexes, lipid nanoparticles, extracellular vesicles and exosome, nucleic acid molecules including DNA such as vector DNA, genomic DNA, antisense oligonucleotides, RNA such as mRNA, siRNA.

Preferably, said biological material is selected from the group consisting of peptides, proteins, protein complexes in combination with nucleic acid molecules include DNA such as vector DNA, genomic DNA, antisense oligonucleotides, RNA such as mRNA, siRNA.

According to an aspect of the invention there is provided a diagnostic method comprising the method according to the invention to measure the integrity of a cell membrane in response to an agent in a cell sample wherein said method comprises the steps:
a) treatment of a cell sample prior to compression with an agent,
b) record the time and pressure until cell lysis, and
c) compare the time and pressure required to lyse the treated cell to the time and pressure required to lyse an untreated cell.

The device according to the invention can be used to diagnose the effect of agents such as drugs and other biological materials on cells by applying pressure onto the cells for a predetermined time until lysis and compare the amount of pressure required to lyse the cell to the amount required to lyse an untreated cell.

Preferably, said cells are compressed at between 0.1-150 kPa, preferably between 1-120 kPa, more preferably between 10-25 kPa, and even more preferably between 15-17.5 kPa and for between 3 to 60 mins, or preferably for 30 min or until the cell is lysed.

According to an aspect of the invention there is provided a method for delivering biological materials to cells in suspension wherein said method comprises the cyclic application of pressure onto the cells to compress said cells to induce uptake of the foreign biological material into the cells.

Preferably, said cyclic application of pressure is repeated, 1, 2, 3, 4, 5, 6, 7, 8, 9 or 10 times.

Preferably said cell is a haematopoietic stem or progenitor cell.

Preferably said mature cell is selected from the group consisting of neutrophils, macrophages, and monocytes/eosinophils, B and T lymphocytes, innate lymphoid cells, dendritic cells, megakaryocytes, or platelets.

Preferably said cell is human cell line HL60.

Preferably said cells are compressed at between 10-25 kPa, preferably between 15-17.5 kPA and even more preferably between 12-17.5 kPa.

Preferably said cells are compressed for between 3 to 10 mins.

Preferably said cells are compressed at 15 kPa for 19 min, or alternatively at 17.5 kPa for 10 min.

Preferably, said biological material is selected from the group consisting of peptides, proteins, protein complexes.

Preferably, said biological material is selected from the group consisting of nucleic acid molecules including DNA such as vector DNA, genomic DNA, antisense oligonucleotides, RNA such as mRNA, siRNA.

Preferably, said biological material is selected from the group consisting of peptides, proteins, protein complexes in combination with nucleic acid molecules including DNA such as vector DNA, genomic DNA, antisense oligonucleotides, RNA such as mRNA, siRNA.

According to an aspect of the invention there is provided a method for the lysis of cells in suspension wherein said method comprises the incremental application of pressure onto the cells until cell lysis.

Preferably, said cells are compressed at between 0.1-150 kPa, preferably between 1-120 kPa, more preferably between 10-25 kPa, and even more preferably between 15-17.5 kPa and for between 3 to 60 mins, or preferably for 30 min or until the cell is lysed.

A method for delivering a pay load or delivering a protein or nucleic acid into mammalian cells in suspension: comprising compressing non-adherent cells cyclically and/or incrementally wherein the step of compressing cells via a valve that can be controlled to mildly deform and undeform the cell membrane (and nucleus) using pressure in cyclical manner and/or incrementally causing perturbation of the cell membrane such that the pay load, protein or nucleic acid enters the cells.

A method of measuring cell membrane integrity of non-adherent mammalian and bacterial cells in suspension: comprising compressing non-adherent cells incremental up to cell lysis, where in the step of compressing cells via a valve can be varied gradually from mild deformation up to cell lysis allowing us to quantify the strength of the cell membrane based on the forces required to destroy the cell membrane Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

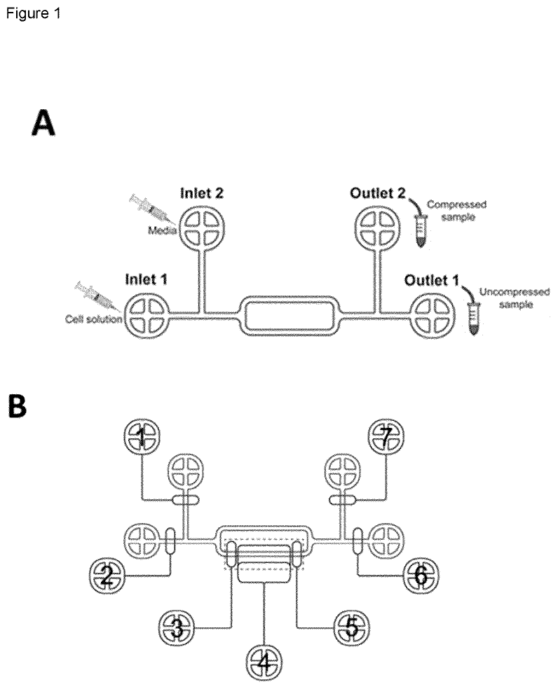

FIG. 1: A trapping unit designed for immobilising and compressing a heterogenous population of cells in suspension. A) A schematic of the two inlet and output mechanisms. B) A schematic of the compressive membrane aligned. C) A photograph of the fabricated device. Control and flow channels are filled with blue and red ink for image capture, respectively. D) BaF3 cells, an immortalised mouse cell line, are introduced into the microfluidics device (scale bar 200 μm). The bottom chamber is used for trapping BaF3 cells as they flow into the device (first valves 1 and 7 were closed prior to loading, in this instance cells were introducing from right to left (as opposed to left to right) through 6a, valve 3 is now closed, valve 6 is then closed, followed by closing valve 5). Once cells are trapped between valves 3 and 5 we begin compression. E) A schematic of the compression chamber. Side view left, plane view right. Cells flow into the bottom layer while the upper layer integrates an air network based on Quake Valve technology (blue). When activated, the latter can selectively compress and obstruct channels of the fluidic layer. F) A heterogenous population of BaF3 cells are subjected to compressive forces by setting the valve at B) 17.5 kPa, 22.5 kPa and 30 KPa (scale bar 50 μm). G) Cell lysis is observed at 35 kPa (scale bar 50 μm).

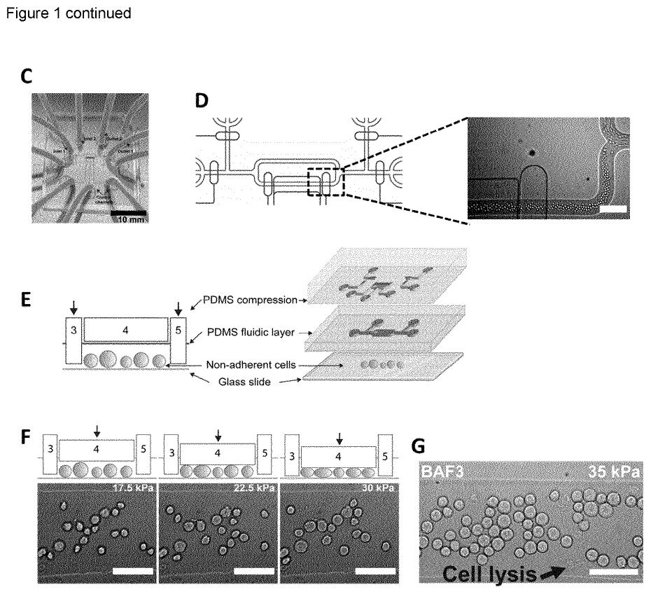

FIG. 2: Device validation using immortalised and primary stem and progenitor cells. A) Between 300 and 500 cells can trapped and compressed simultaneously in the compression chamber (e.g. HL60 a human leukemic cell line, scale bar 200 μm). B) A heterogenous population of BaF3 cells are immobilised and no deformation is observed at 12.5 KPa (C) Larger HL60 cells on the other hand are deformed when the compression chamber is set at the same pressure D) TET2 mutated mouse HSCs are deformed at 25 kPa for 10 mins. E) Wild type mouse GMPs are deformed at 22.5 kPa for 50 mins. Scale bar 50 μm. F) A density histogram of the spread area of TET2+/−GMPs pre-compression (red) and post-compression at 25 kPa (green) and 28 kPa (blue). Retrieval of (G) wild type GMPs following 60 min compression (22.5 KPa) with overnight culture demonstrating that the device can apply forces without compromising cell survival. Scale bar 50 μm.

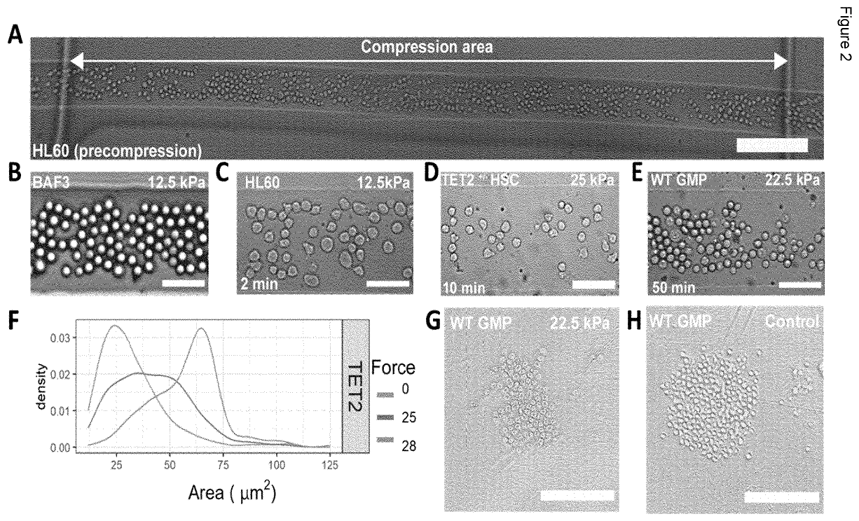

FIG. 3: Compressive stresses do not affect the viability of haematopoietic progenitor cells, but affects the mature cell output. A) Wild type and TET2-deficient mouse GMPs were compressed for 30 mins at 22.5 kPa (n=6, TET2, and n=6 WT), 28 kPa (n=4 TET2, n=8 WT) and for 60 mins at 20 kPa (n=9 TET2, n=1 WT). For each independent experiment three control samples were collected and analysed per condition B) Wild type and TET2-deficient GMPs were compressed for 30mins at 22.5 kPa (n=3, TET2, and n=2 WT), 28 kPa (n=4 TET2, n=5 WT) and for 60 mins at 20 kPa (n=8 TET2, n=1 WT). Following 10 days of culture, cells were analysed by flow cytometry to measure the impact of compressive stresses on the differentiation capacity of progenitor cells. The application of compressive forces in our chamber for 60 mins at 20 Kpa has resulted in cells with increased cKit expression (i.e. these cells are more primitive) in TET2-deficient GMPs (***p<0.001, n=8 independent experiments) . As in FIG. 2, this demonstrates that the compressive forces do not compromise stem and progenitor cell function or viability.

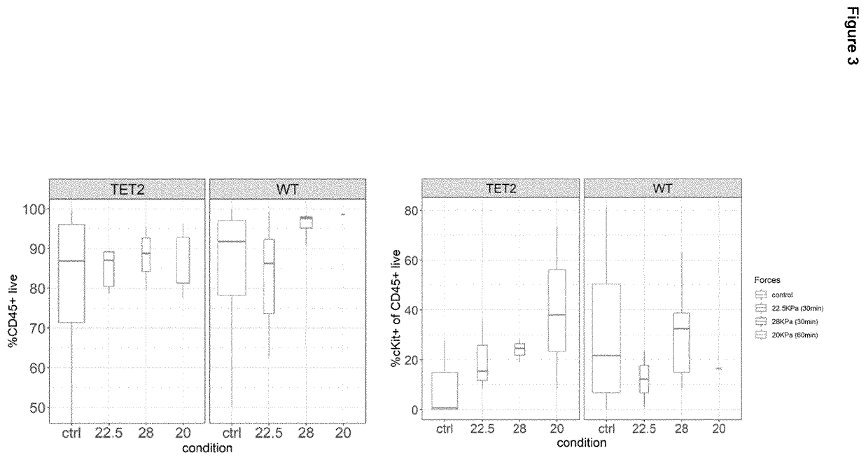

FIG. 4: Compressive forces do not affect the viability of Haematopoietic stem cells but may affect mature cell output. Primary EPCR+Lin-Sca 1+cKit+(ELSK) HSCs isolated from 28-day expansion cultures were compressed for 20 mins at 28.5 kPa (n=1) and for 30 mins at 25 kPa (n=3). Following 10 days of culture, cells were analysed by flow cytometry to measure the impact of compressive stresses on cell viability in vitro, and how compressive forces affect the differentiation capacity of progenitor cells. Cell viability and the output of immature and mature myeloid cells is represented by staining for mature cell markers A) viability and mature cell output is not affected when 25 kPa is applied for 30 mins. Output of immature and mature myeloid cells is represented by staining for mature cell markers B) % Lin-Sca+cKit+ (LSK) C) % ELSK D) % Lin−. E) When 28 kPa is applied for 20 mins, viability is also not affected but mature output is altered as seen in F) % Lin− cells and G) % LSK cells in TET2-deficient cells.

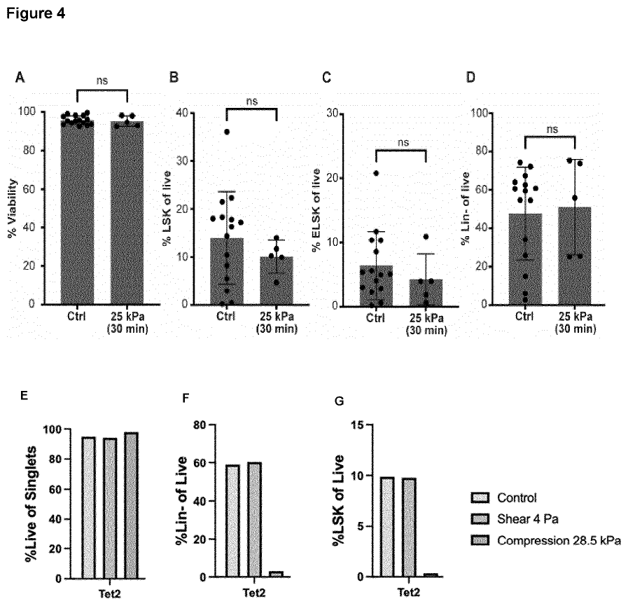

FIG. 5: Compression enhances plasmid-GFP transduction efficiency. Plasmid DNA (pF-GFP) uptake at [0.5 ng] A) Bright field HL60 18 hours post transduction using DNA transfection reagent jetOPTIMUS B) GFP fluorescence images C) Bright field HL60 18 hours post compression (cyclic compression ranging from 12.5 kPa up 17.5 kPa for 20 mins) D) GFP fluorescence.

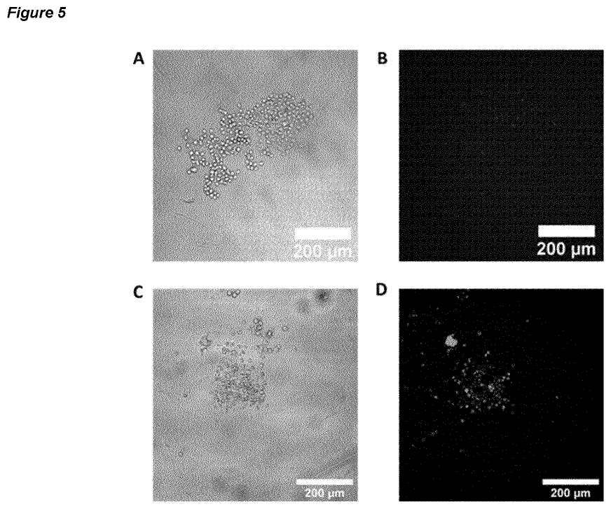

FIG. 6: Gram positive and Gram negative trapped in the compression chamber:
A) Around 2000 E. coli are trapped (precompression)
B) Staphylococcus aureus trapped (precompression)
C) Staphylococcus aureus (untreated) post compression where cells spread area changed, and lysis was obtained under 90 kPa.

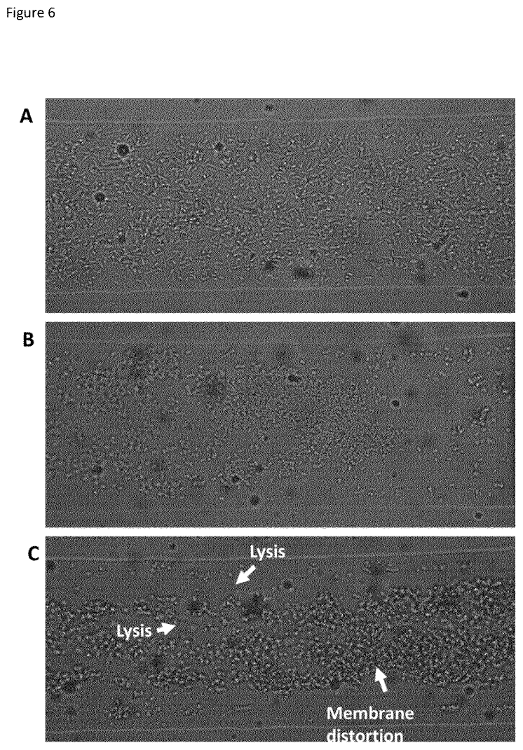

FIG. 7—Schematic of Microfluidic device

### MATERIALS AND METHODS

### Chip Design and Fabrication

Using AutoCAD (Autodesk Inc.) we designed a two-layer chip (a flow and a control layer) and printed chrome masks at 50 kdpi resolution (JD photo, UK). Each chip consisted of a parallel channel with two inflow and outflow mechanisms. The microfluidic device was fabricated according to standard photolithography and soft lithography procedures. Specifically, the flow layer was achieved by depositing and reflowing AZ-40XT (MicroChem) to obtain an 11-16 μm parabolic surface at valve positions. AZ-40XT was spin-coated for 60 s at 4500 rpm before soft baking 5 min at 65° C. and 10 min at 115° C. After cooling to room temperature, the master template was exposed to UV light (120 mJ/cm²) through the photomask with a MA70 mask aligner (OAI, Optical Associates Inc.) before baking 5 min at 65° C. and 10 min at 110° C. Master templates were then developed for 120 s in MIF 726 (MicroChem). In order to reflow the channels up to 100 micron wide features, the templates were then baked for 8 min at 110° C. and 120° C. for 15 min.

To obtain a 1-2 μm parabolic surface at valve positions. AZ 3027 was spin-coated for 60 seconds at 4000 rpm before soft backing 90 seconds at 95° C. After cooling to room temperature, the master template was exposed to UV light (262 mJ/cm²) through the photomask with a MA70 mask aligner (OAI, Optical Associates Inc.) before baking 1 min at 95° C. and 2 min at 125° C. Master templates were then developed for 55 s in MIF 726 (MicroChem). In order to reflow the channels up to 1-2 micron wide features, the templates were then baked for 3 minutes at 120° C. and 130° C. for 5 min.

The control layer was achieved by depositing and reflowing a negative epoxy photoresist SU8-2025 (kayaku AM). SU8-2025 was spin-coated for 60 s at 4100 rpm before soft baking for 1 min and 30 s at 65° C. and 3 min and 30 s at 95° C. After cooling to room temperature, the master template was exposed to UV light (140 mJ/cm²) through the photomask with MA70 mask aligner (OAI, Optical Associates Inc.) before baking for 1 min at 65° C. and 5 min at 95° C. Master templates were then developed for 120 s in SU-8 developer 4 poly (Kayaku AM). Hard bake for 3 minutes at 65° C. and 5 minutes at 95° C. Features obtained were 18 microns high and up to 20 microns wide. Templates were further hard baked for 1 hour at 190° C.

After producing our silicon moulds (master templates), we mixed and degassed 50g of Poly(dimethylsiloxane) (PDMS; Sylgard 184; Dow-Corning) (5:1; polymer:catalyst) before coating the SU8 control layer/master template and curing for 15 min at 80° C. Similarly, we poured 20 g of mixed and degassed PDMS (20:1; polymer:catalyst) over the AZ flow layer/master template before spinning at 2200 rpm for 45 s and curing for 15 min at 80° C. Stamps were removed from the SU8 master templates and inlet holes were punched (1.5 mm punch). Partially cured PDMS stamps were then aligned to the flow layer master template. After 5 h of thermal bonding at 80° C., the PDMS stamps were then carefully peeled off from the AZ master template. Channels inlets and outlets were then punched (1 mm punch). Stamps were plasma treated before bonding to glass coverslips. We then cured the completed chip for 15 min at 80° C., and submerged devices into sterile water for 3-4 hours.

### 2. Chip Control Set-up

The first inlet in the microfluidics device is connected to a 1 ml syringe pump (certified neMESYS low pressure syringe pump system, Cetoni GmbH, Korbussen, Germany, NEM-B101-03 A with a gear ratio of 29:1). The second inlet and the two outlets are connected to 1 mm PTFE (Polytetrafluoroethylene) tubes (of inner and outer diameter of 0.72 and 1 mm, respectively) which are connected to an Eppendorf tube.

For primary granulocyte-macrophage progenitor cell (GMP) experiments. The device is perfused with medium (Iscove's Modified Dulbecco's Medium (IMDM) supplemented with foetal calf serum (FCS), L-glutamine, penicillin/streptomycin and beta-mercaptoethanol) at 1.5 μL/minute for between 15 and 30 minutes where the media flow throughout the device and exit from the device from all 3 PTFE tubes connected to Eppendorf tubes. For cell nucleotide delivery experiments, the device is perfused with OptiMEM media which included locked nucleic acid LNA (25 nM). Once the media exits from all tubes, the second inlet is connected to a separate 1 ml syringe pump (certified neMESYS low pressure syringe pump system (Cetoni GmbH, Korbussen, Germany), and the previous PTFE tube is discarded.

The microfluidic chip valves are connected to airtight tubes via tubing with the inner and outer diameter of 0.9 and 1.6 mm, respectively. Valves 1, 2, 3, 5, 6, 7 are controlled by a microfluidic valve control matrix (MUX QUAKE VALVE, Elveflow). Valve 4 is controlled by a separate/second microfluidic valve control matrix (MUX QUAKE VALVE, Elveflow). Both MUX QUAKE Elveflow are connected to Elveflow pressure and vacuum controller pump (model OB1 Mk3 with channel pressure up to 8000 mbar, and 2000 mbar respectively). The control matrix is programmed by a PC. The pressure controller (ELVEFLOW® OB1 Mk3) is used to precisely regulate the pressure to either activate or deactivate the pneumatic microvalves

### 3. Chip Preparation on Microscope

This chip/device is visualised using Leica DMI3000 consisting of ORCA Flash 4.0 of Hamamatsu Photonics. Images were acquired by an in-house developed LabVIEW software. All valves are tested and checked (activate or deactivate the pneumatic microvalves) prior to loading the cells.

### 4. Preparation of Cell Suspension and Loading of Cells Onto the Chip

Primary GMP experiments are performed using freshly isolated primary cells (cells were harvested from mouse bone marrow as described in the literature [1]. Between 200,000 and 500,000 GMPs are centrifuged in Terasaki plates for 5 minutes at 240×g, and placed on ice. For haematopoietic stem and progenitor cell (HSC) experiments, between 50,000 and 150,000 cells are centrifuged in Terasaki plates for 5 minutes at 300×g, and placed on ice. For cell nucleotide delivery experiments delivery experiments 300,000 cells of Human HL60 cells in RPMI1640 media (10% FBS, 1% PenStrep Glutamine) are centrifuged in Terasaki plates for 5 minutes at 300×g, at room temperature. Devices are already loaded with OptiMEM media which included locked nucleic acid LNA (25 nM).

Prior to loading the cells into the device, pneumatic pressure is applied to valve 1 and valve 7 at 80 kPa (using the first MUX QUAKE VALVE) to prevent any liquid flow into the second inlet and outlet. Cells are loaded into 1 mL syringes through a PTFE tube of inner and outer diameter of 0.72 and 1 mm, respectively. Cells are loaded by aspirating them from Takasaki plates at rate of 18 μl/min). Cells are perfused into the devices at 1.5 μL/min between 1 min-5 min until cells are trapped in the compression chamber. Once cells are trapped, perfusion is stopped and pneumatic pressure is applied to valve 2 at 80 kPa (using the first MUX QUAKE VALVE) to prevent any extra fluid flow into the device.

For Antibiotics sensitivity experiments: experiments were performed using Gram-negative (e.g Escherichia coli) and Gram-positive (e.g Staphylococcus aureus) bacteria. Bacterial cultures were grown overnight in a suitable growth medium at 37° C. with shaking. Conditions for antibiotic susceptibility testing were adapted from Kowalska-Krochmal & Dudek-Wicher, 2021. Cells were then diluted 1:100 into Mueller-Hinton (MH) broth supplemented with 2% NaCl and allowed to grow until they enter exponential growth (optical density between 0.2 and 0.5 AU). Cells were then concentrated down to a final cell density of 5×10⁹ cells ml⁻¹. Samples were spiked with 10 μg/ml ampicillin and incubated at 37° C. with shaking for 30 mins before transferring the cells to ice.

Prior to loading the cells into the device, pneumatic pressure is applied to valve 1 and valve 7 at 90 kPa (using the first MUX QUAKE VALVE) to prevent any liquid flow into the second inlet and outlet. Cells are loaded into 1 mL syringes through a PTFE tube of inner and outer diameter of 0.72 and 1 mm, respectively. Cells are loaded by aspirating them from Eppendorf tube at rate of 18 μl/min). Cells are perfused into the devices at 1.5 μL/min between 1 min-5 min until cells are trapped in the compression chamber (both Gram positive and Gram Negative). Once cells are trapped, perfusion is stopped, and pneumatic pressure is applied to valve 2 at 100 kPa (using the first MUX QUAKE VALVE) to prevent any extra fluid flow into the device.

### 5. Conducting a Cell Mechanical Stimulation Experiment

### Cell Entrapment

Prior to loading the cells. Valves 1 and 7 are partially closed to avoid cells entering/existing in the second input/out path (FIG. 1A). During cell perfusion, while mammalian cells flow in the bottom chamber (FIG. 1B, highlighted area). First pneumatic pressure is applied to valve 5 at 80 kPa (using the first MUX QUAKE VALVE) to prevent cells from exiting the compression chamber (FIG. 1D). Once cells are immobilised in the bottom chamber, pneumatic pressure is applied on valve 3 at 80 kPa (using the first MUX QUAKE VALVE) to trap the cells in the bottom chamber between valve 3 and valve 5. Extra flow is stopped via valve 2 as mentioned above.

Pneumatic pressure is removed from valve 1, and media is perfused using the second inlet at 1.5 L/min. Media perfusion removes all cells from the device except those trapped in the bottom chamber. Once cells exit the device, perfusion is stopped and pneumatic pressure is applied to valve 1 at 80 kPa (using the first MUX QUAKE VALVE) to prevent any further media flow.

### Incremental and Cyclic Compression Assays

Pneumatic pressure (using the second MUX QUAKE VALVE) is applied ranging from mild deformation through to complete cell lysis (e.g. FIG. 1F-1G). For GMP experiments, pressures of 22.5 kPa, 25 kPa, 28 kPa were chosen for the duration of 30 minutes and 60 minutes. For HSC experiments, pressures of 25 kPa, and 28.5 kPa were chosen for the duration of 20 minutes and 30 minutes. For cell nucleotide delivery experiments, pressured of 12 kPa and 17.5 kPa were chosen for 10 and 3mins respectively. We also found that cyclic compression has enhances plasmid-GFP delivery efficiency in HL60 cells (FIG. 5). For cyclic compression, while valves 1, 2, 3, 5 and 7 are partially closed, valve 4 is alternating between open and partially closed. We can also allow valve 3 to occasionally open, after opening valve 4, this allows extra media and nucleic material to flow inside the compression chamber. We incubate the cells inside the chamber for 5 mins before closing valve 3 then compressing using membrane/valve 4 again for 1-2 mins.

### 6. Retrieving Cells From the Chip for Viability and Maturation Analysis

Shear samples are collected in the Eppendorf connected to the first outlet. The second outlet will be used to collect the compression sample.

At the end of the compression assay, a pneumatic pressure is applied to valve 6 at 80 kPa (using the first MUX QUAKE VALVE) and the pneumatic pressure is removed from valve 7 closing outlet 1 and opening outlet 2 respectively. To release the cells into the collection tube (Eppendorf connected to second outlet), pneumatic pressure is removed from valve 1, and media is perfused at 1.5 μL/min, pneumatic pressure is removed from valve 5, 4 and 3 sequentially, where cells are directed to the collection tube.

### 7. Retrieving Cells From the Chip for Gene Expression Analysis

Samples are collected into an Eppendorf tube and an RNA lysis buffer+RNAse inhibitor is added before freezing samples that can be subsequently be processed for transcriptional profiling (e.g., RNA-sequencing, qPCR, etc).

### 8. Retrieving Cells for Cell Nucleotide Delivery Experiments

Samples are collected into collected into an Eppendorf tube and cultured in RPMI1640 media (10% FBS, 1% PenStrep Glutamine) for 48 hours then processed for qPCR.

### EXAMPLE 1

A novel microfluidic device capable of mechanically stimulating a range of isolated haematopoietic stem and progenitor cell (HSPC) fractions.

The advantage of this technology is in the ability to immobilise (FIG. 1D, FIG. 2A) and apply quantifiable compressive stresses (FIG. 2B-2E B-1F) on a population of cells, for example HSPCs, in liquid suspension. The device is also capable of applying a static compressive stress for a prolonged period of time (e.g. FIGS. 2D, 2E) ranging from mild deformation through to complete cell lysis (FIG. 1G. Cells can then be retrieved for cell culture (e.g. FIG. 2G, 2H), and for functional and molecular analysis. This device fills the tool gap described above and allows us to physically compress non-adherent cells and study the impact of physical forces on cellular fate and to explore potential applications for therapeutic cargo delivery.

### EXAMPLE 2

### Determination of the Forces Cells Can Tolerate Without Losing Functionality

Our preliminary experiments using 10-day cultures with assessment of cell type by flow cytometry shows that compression can alter the output of mature myeloid cells (e.g. cKit expression, 3B)) without compromising cell viability (FIG. 3A, FIGS. 4A, 4E), suggesting that mechanical signals can alter stem and progenitor cell fate in the absence of other changes.

Specifically, we can see that for TET2-deficient GMPs, the application of compressive forces in our chamber for 60 mins at 20 Kpa has significantly increased retention of cKit expression on stem/progenitor cells (***p<0.001, n=8 independent experiments, FIG. 3B)

### EXAMPLE 3

### Determine How Cell Compression Can Manipulate Stem Cell Fate

Experiments using HSCs from 28-day expansion cultures [5, 6] show that compressive stresses can alter TET2-deficient HSCs when compressed for a period of just 20 mins (FIGS. 4F, 4G). Millions of cells are created in these HSC expansion conditions, and the vast majority of cells are non-HSCs despite the increased absolute number of HSCs. The broad maturation state of the populations of cells comprising the cultures is very easily monitored by flow cytometry and it is clear from FIG. 4 that compressive stresses compression can alter the speed at which cells leave the immature state.

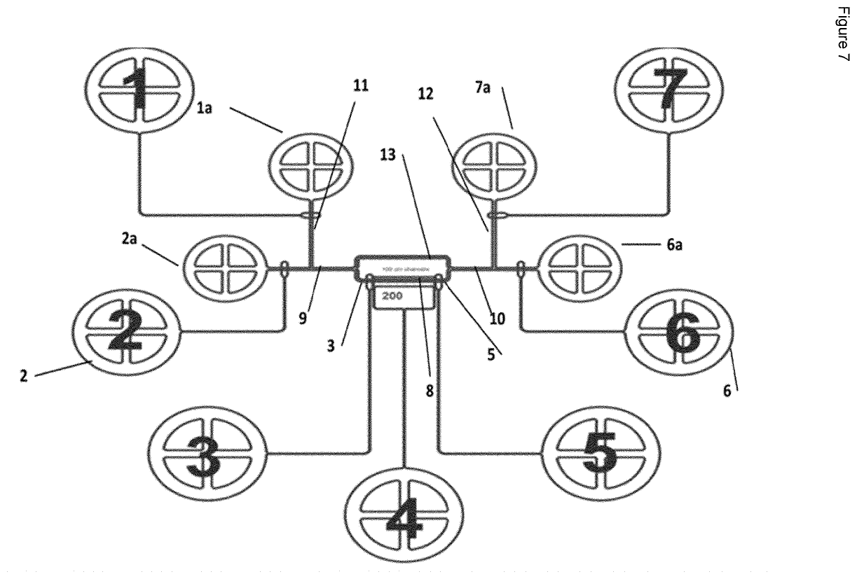

FIGS. 3 and 44 suggests that compressive stress can play a role in directing the cell fate of HSCs isolated from expansion cultures. This opens the opportunity to explore the potential for mechanical signalling to direct the maturation of HSCs in expansion cultures, which creates new opportunities in producing cell types in vitro using mechanical stimuli.

### 3. Cell Compression As a Tool for Improved Cargo Delivery

Numerous cell and gene therapy applications require the delivery of different molecules to cells and, in many cases, protocols are limited by efficient delivery of those molecules (in particular larger molecules). Haematopoietic stem cells are particularly difficult to deliver molecules to (e.g. viral transduction efficiencies in primitive stem cells are often <10%;)). Moreover, recent delivery approaches involving peptides, proteins, protein complexes, and nucleic acid delivery are all hampered by an inability to move across membranes (cell or nuclear). Incremental and cyclic cell compression during the delivery phase offers a novel approach to deliver these molecules due to temporary, non-toxic, and controllable changes in the cell membrane. Current approaches (e.g., electroporation, nucleofection, etc) typically requires large numbers of cells and often results in rupturing the cells completely. Our device would be able to permit precise control of both the amount and duration of compression on specific target cell populations to achieve changes in a much more precise manner.

As an example we have used the microfluidic device to deliver plasmid-GFP into HL60 (an acute promyelocytic leukaemia suspension cell line). Cyclic compression has increased plasmid DNA (pF-GFP) uptake at [0.5 ng] with up to 47.6% efficiency (FIGS. 5C, 5D). The use of DNA transfection reagent jetOPTIMUS alone (FIGS. 5A, 5B) resulted in transfection efficiencies of less than 2%. The delivery in the HL60 system is known to be difficult and these new results therefore demonstrate the potential applications for gene editing.

### 4. Cell Compression as a Tool for Measuring Cell Membrane Integrity

We are capable of capturing and deforming (all the way to cell lysis) cells in suspension. This permits measurement of cell integrity as individual molecules or combinations of molecules are added. The efficacy of antibiotics that aim to kill bacteria by damaging their cell membranes can be tested rapidly. As an example we have captured both gram positive and gram negative cells in our chip (FIGS. 6A, 6B) and without adding any antibiotics and or molecules to weaken the cell membrane we were able to distort and lysis the cell membrane at higher compressive stresses (FIG. 6C).

### REFERENCES

1. Nestorowa, S., et al., A single-cell resolution map of mouse hematopoietic stem and progenitor cell differentiation. Blood, The Journal of the American Society of Hematology, 2016. 128(8): p. e 20-e31.

2. Guo, Q., et al., Microfluidic biomechanical assay for red blood cells parasitized by Plasmodium falciparum. Lab on a Chip, 2012. 12(6): p. 1143-1150.

3. Gossett, D.R., et al., Hydrodynamic stretching of single cells for large population mechanical phenotyping. Proceedings of the National Academy of Sciences, 2012. 109(20): p. 7630-7635.

4. Vining, K.H. and D.J. Mooney, Mechanical forces direct stem cell behaviour in development and regeneration. Nat Rev Mol Cell Biol, 2017. 18(12): p. 728-742.

5. Wilkinson, A.C., et al., Long-term ex vivo haematopoietic-stem-cell expansion allows nonconditioned transplantation. Nature, 2019. 571(7763): p. 117-121.

6. Che, J.L.C., et al., Identification and characterization of in vitro expanded hematopoietic stem cells. EMBO Rep, 2022: p. e55502.

7. DiTommaso, T., et al., Cell engineering with microfluidic squeezing preserves functionality of primary immune cells in vivo. Proceedings of the National Academy of Sciences, 2018. 115(46): p. E10907-E10914.

8. Kowalska-Krochmal & Dudek-Wicher, 2021

1. A microfluidic device comprising a compression chamber (4,8) comprising a chamber (8) and a compression membrane (4) adapted to contain and compress cells contained therein, wherein said compression chamber (4, 8) is provided with at least first and second channels (9, 10) in fluid connection with said compression chamber (4. 8) wherein said first channel (9) is provided with an inlet (2a) at or near the end of said first channel (9) for receiving cells and further comprising a first valve (3) to regulate the flow of cells from said inlet (2a) to said compression chamber (4, 8 ) via said first channel (9) and wherein said second channel (10) is provided with an outlet (6a) at or near the end of said second channel (10) to regulate, when in use, the exit of compressed cells contained in said compression chamber (4, 8) and further comprising a second valve (5) to regulate the flow of compressed cells from said compression chamber (4,8) via said second channel (10) wherein said first and second valves (3, 5) are independently regulatable to control the ingress and egress of cells respectively into and out of said compression chamber (4, 8), wherein there is provided a fifth channel (13) in fluid connection with the first (9) and second channel (10) to receive a fluid and/or a fluid comprising cells during compression.

2. The microfluidic device according to claim 1 wherein said microfluidic device comprises a silicone-based polymer, optionally poly(dimethylsiloxane) (PDMS).

3. The microfluidic device according to claims 1 or 2 wherein said membrane has an upper and lower surface and a space exist between the upper and lower membrane for receiving a fluid.

4. The microfluidic device according to any one of claims 1 to 3 wherein said membrane has a width and the width of the membrane exceeds the width of the chamber.

5. The microfluidic device according to any one of claims 1 to 4 wherein said chamber has a length and the length of the chamber exceeds the length of the membrane.

6. The microfluidic device according to any one of claims 1 to 5 wherein said first channel comprises a further valve (2) to regulate the flow from the first inlet (2a) into the first channel (9).

7. The microfluidic device according to any one of claims 1 to 6 wherein said second channel comprises a further valve (6) to regulate the flow towards the second channel (10) into the first outlet (6a).

8. The microfluidic device according to any one of claims 1 to 7 wherein said first channel (9) is in fluid connection with a third channel (11) and which is provided with a second inlet (1a) at or near the end of said third channel (11) for receiving cells or liquids.

9. The microfluidic device according to any one of claims 1 to 8 wherein said third channel (11) is provided with a valve (1) to regulate the flow from the second inlet (1a) to the first channel (9) and compression chamber (8,4).

10. The microfluidic device according to any one of claims 1 to 9 wherein said second channel (10) is in fluid connection with a fourth channel (12) and provided with a second outlet (7a) at or near the end of said fourth channel (12).

11. The microfluidic device according to any one of claims 1 to 10 wherein said fourth channel (12) is provided with a valve (7) to regulate the flow from the compression chamber (4,8) and the second channel (12) to the second outlet (7a).

12. The microfluidic device according to any one of claims 1 to 11 wherein said fifth channel (13) is positioned substantially in parallel with the compression chamber (4,8).

13. The microfluidic device according to any one of claims 1 to 12 wherein said first, second, third, fourth and fifth channel are 2 to 35 μm in height.

14. The microfluidic device according to any one of claims 1 to 13 wherein said first, second, third, fourth and fifth channel are 16 μm±5 μm in height.

15. The microfluidic device according to any one of claims 1 to 14 wherein said first, second, third, fourth and fifth channels are between 40 μm+/10% to 400 μm+/10% in width.

16. The microfluidic device according to any one of claims 1 to 15 wherein said first, second, third, fourth and fifth channel are 100 μm+/10% in width and 16 μm±5 μm in height.

17. The microfluidic device according to claims 1 to 15 wherein the height of the first, second, third, fourth, fifth channel and the chamber is the same.

18. The microfluidic device according to any one of claims 1 to 17 wherein said compression chamber is 100 μm+/10% in width and 16 μm±5 μm in height.

19. The microfluidic device according to any one of claims 1 to 18 wherein said compression chamber is not adapted by the provision of a cell attachment surface, for example said compression chamber is not coated with cell attachment proteins such as fibronectin.

20. The microfluidic device according to any one of claims 1 to 19 wherein said device comprises cells.

21. A method for the compression of cells comprising the steps:
i) providing a microfluidic device comprising a compression chamber comprising a chamber (8) and a compression membrane (4) adapted to contain and compress cells contained therein, wherein said compression chamber (4, 8) is provided with at least first and second channels (9, 10) in fluid connection with said compression chamber (4. 8) wherein said first channel (9) is provided with an inlet (2a) at or near the end of said first channel (9) for receiving cells and further comprising a first valve (3) to regulate the flow of cells from said inlet (2a) to said compression chamber (4, 8 ) via said first channel (9) and wherein said second channel (10) is provided with an outlet (6a) at or near the end of said second channel (10) to regulate, when in use, the exit of compressed cells contained in said compression chamber (4, 8) and further comprising a second valve (5) to regulate the flow of compressed cells from said compression chamber (4,8) via said second channel (10) wherein said first and second valves (3, 5) are independently regulatable to control the ingress and egress of cells respectively into and out of said compression chamber (4, 8); and a fifth channel (13) in fluid connection with the first (9) and second channel (10) to receive a fluid and/or a fluid comprising cells during compression.
ii) feeding a cell sample to be compressed in said first channel (9) via said inlet (2a) into said compression chamber (4, 8); and partially close said second valve (5) to maintain flow of fluids but prevent movement of cells;
iii) partially closing valve (2);
iv) partially close said first valve (3) to maintain flow of fluids but prevent movement of cells thereby containing said cell sample in said compression chamber (4, 8);
v) actuating said compression membrane (4) of said compression chamber (8) thereby deflecting said compression membrane (4) and imparting compression force on said chamber (8) containing said cell sample to compress cells contained therein;
vi) opening valve (2) and feeding buffer into said first channel (9) via said inlet (2a) into said compression chamber (4, 8);
vii) open said second valve (5), actuating said membrane (4) of said compression chamber (4, 8) thereby deflecting said membrane and removing compression forces on said chamber (8) applied in step v) and open said first valve (3) to allow egress of compressed cells from said compression chamber into said second channel (10); and
viii) collecting said compressed cells via outlet (6a).

22. The method according to claim 21 wherein said first channel under i) comprises a further valve (2) to regulate the flow from the first inlet (2a) into the first channel (9),

23. The method according to claims 21-22 wherein said microfluidic device comprises further a third channel (11), wherein the third channel (11) in fluid connection with said first channel (9) and is provided with a third valve (1) and a second inlet (1a) at or near the end of said third channel (11) and wherein said third valve (1) regulates the flow from the second inlet (1a) to the first microfluidic channel (9) and compression chamber (4,8) and
a fourth channel (12), wherein the fourth channel (12) in fluid connection with said second channel (10) and is provided with a fourth valve (7) and a second outlet (7a) at or near the end of said fourth channel (12) and wherein said fourth valve (7) regulates the flow from the compression chamber (4,8) and second microfluidic channel (10) via the fourth channel (12) to the second outlet (7a).
and wherein step ii) includes closing of valve (1) and (7) prior feeding a cell sample to be compressed
and wherein step iv) after partially closing valve (3) further comprises opening valve (1) and feeding buffer via inlet (1a) in said third (11), first (9) and fifth (13) channel to allow removal of non-compressed cells via the second cannel (10) and first outlet (6a) and wherein valve (7) is closed; and wherein step viii) alternatively comprises closing valve (6) and opening valve (7) to collect compressed cells via the second outlet (7a).

24. The method according to any one of claims 21 to 23 wherein said step v) comprises further the step v1: actuating said compression membrane (4) of said compression chamber (4,8) thereby deflecting said membrane and removing compression forces on said chamber (8) followed by actuating said compression membrane (4) of said compression chamber (8) thereby deflecting said membrane and imparting compression forces to provide cyclic compression on said cells.

25. The method according to claim 24 wherein said step v1 is repeated 1, 2, 3, 4 or 5 times.

26. The method according to claims 24 or 25 wherein said deflection of the membrane to impart compression in step v1 is for a length of 1, 2, 3, 4, 5, 6, 7, 8, 9, 10, 11, 12, 13, 14, 15, 16, 17, 18, 19, 20, 25 or 30 min.

27. The method according to claims 24 or 25 wherein said deflection of the membrane to remove compression in step v1 is for a length 1, 2, 3, 4, 5, 6, 7, 8, 9, 10, 11, 12, 13, 14, 15, 16, 17, 18, 19, 5, 10, 15, 20, 25 or 30 min.

28. The method according to any one of claims 24-27 wherein said compression forces are increased during each compression cycle.

29. The method according to any one of claims 24-28 wherein said compression force is sequentially increased from a starting force of 0.1 kPa up to between 100-250 kPa.

30. The method according to claim 29 wherein said compression force is sequentially increased from a starting force of 0.1 kPa up to between 150-190 kPa.

31. The method according to any one of claims 21 to 30 wherein said cell sample comprises cells in a cell suspension.

32. The method according to claim 31 wherein said cells are a suspension of heterogeneous cells.

33. The method according to claim 31 wherein said cells are a suspension of homogeneous cells.

34. The method according to any one of claims 21 to 33 wherein said cells are non-adherent cells.

35. The method according to any one of claims 21 to 34 wherein said cells are mature cells.

36. The method according to any one of claims 21 to 34 wherein said cells are immature/progenitor cells.

37. The method according to claim 36 wherein said immature/progenitor cell selected from the group consisting of: multi-potent progenitors (MPPs); lympho-myeloid primed progenitors (LMPPs); common myeloid progenitors (CMPs); granulocyte-macrophage progenitors (GMPs); or megakaryocyte-erythrocyte progenitors (MEPs).

38. The method according to any one of claims 21 to 37 wherein said compression force is provided between 5-30 kPa, preferably between 10-28.5 kPa, more preferable between 20-28 kPa.

39. The method according to any one of claims 21 to 38 wherein said compression force is applied for between 10-60 min and more preferably between 20-50 min or 30-40 min.

40. The method according to any one of claims 21 to 39 wherein said microfluidic device is linked to a graphical user interface to control the compression and visually monitor the cells during compression.

41. Cells obtained by the method according to claims 21 to 40.

42. Use of the microfluidic device according to claims 1 to 20 for the delivery of biological materials to a cell.

43. Use the microfluidic device according to claims 1 to 20 for cell maturation.

44. A method for inducing maturation of an immature cell into a mature cell comprising the method according to clams 21 to 40.

45. The method according to claim 44 wherein said immature cell is a haematopoietic stem cell, and optionally said mature cell is selected from the group consisting of neutrophils, macrophages, monocytes, basophils, eosinophils, B and T lymphocytes, innate lymphoid cells, dendritic cells, megakaryocytes, or platelets.

46. The method according to claims 44 or 45 wherein said immature cells are compressed at between 20-28 kPa for between 30-60 min.

47. A method for delivering biological materials into cells comprising the method according to claims 21-40, wherein said cell sample comprises biological material to be delivered into the cell.

48. The method according to claim 47 wherein said biological material is selected from the group consisting of peptides, proteins, protein complexes, lipid nanoparticle, extracellular vesicle or exosomes.

49. The method according to claim 47 wherein said biological material is selected from the group consisting of nucleic acid molecules including DNA such as vector DNA, genomic DNA, antisense oligonucleotides, RNA such as mRNA, siRNA.

50. Use of the microfluidic device according to any one of claims 1-20 for cell lysis.

51. Use of the microfluidic device according to any one of claims 1-20 for measuring cell membrane integrity.

52. A diagnostic method comprising the method according to anyone of claims 21-40 to measure the integrity of a cell membrane in response to an agent in a cell sample wherein said method comprises the steps:
a) treatment of a cell sample prior to compression with an agent,
b) record the time and pressure until cell lysis, and
c) compare the time and pressure required to lyse the treated cell to the time and pressure required to lyse an untreated cell.

53. A method for delivering biological materials to cells in suspension wherein said method comprises the cyclic application of pressure onto the cells to compress said cells to induce uptake of the biological material into the cells.

54. The method according to claim 53 wherein said biological material is selected from the group consisting of peptides, proteins, protein complexes lipid nanoparticle, extracellular vesicle or exosomes, nucleic acid molecules including DNA such as vector DNA, genomic DNA and antisense oligonucleotides, RNA such as mRNA or siRNA.

55. The method according to claims 53-54 wherein said cells are compressed at between 10-25 kPa, preferably between 15-17.5 kPa for between 3 to 10 mins.

56. A method for lysing cells in suspension comprising the incremental application of pressure onto the cells to compress the cells until cell lysis.

57. the method according to claim 56 wherein pressure is applied onto the cells at between 0.1-150 kPa.

58. The method according to claims 56 or 57 wherein said cells are compressed for between 3 to 60 mins.
